(19) United States
(12) Patent Application Publication
(10) Pub. No.: US 2017/0372011 A1
(43) Pub. Date: Dec. 28, 2017

(54) METHOD FOR PROVIDING A MEDICAL INFORMATION SYSTEM, AND MOTOR VEHICLE

(71) Applicant: Audi AG, Ingolstadt (DE)

(72) Inventor: Elmar NOETH, Ingolstadt (DE)

(73) Assignee: Audi AG, Ingolstadt (DE)

(21) Appl. No.: 15/542,385

(22) PCT Filed: Jan. 11, 2016
(86) PCT No.: PCT/EP2016/000033

§ 371 Date: Jul. 7, 2017

(30) Foreign Application Priority Data
Jan. 13, 2015 (DE) 10 2015 000 401.8

(51) Int. Cl. G06F 19/00; A61B 5/18; A61B 5/00

(52) U.S. Cl. CPC G06F 19/322; G06F 19/3418; A61B 5/0015; A61B 5/18; A61B 5/686

(57) ABSTRACT

The invention relates to a method for providing a medical information of a vehicle occupant of a motor vehicle and/or of a medical device of the vehicle occupant, in which a signal comprising the medical information is transferred wirelessly to equipment arranged in the motor vehicle, wherein the medical information is transmitted wirelessly from the equipment to the apparatus external to the motor vehicle.

### TECHNICAL FIELD

The invention relates to a method for providing medical information from a vehicle occupant of a motor vehicle and/or of a medical device of the vehicle occupant, in which a signal comprising the medical information is transferred wirelessly to equipment arranged in the motor vehicle. The invention moreover relates to a system as well as a motor vehicle.

### BACKGROUND

Methods for providing medical information of a vehicle occupant of a motor vehicle and/or of a medical device of the vehicle occupant are known from the prior art. In DE 10 2011 016 776 A1, a method is thus described in which health status data of a vehicle occupant are determined by means of a medical device and are transmitted from the medical device to the motor vehicle by means of a data transmission.

### SUMMARY

It is an object of the invention to provide a method, a system and a motor vehicle with which or in which a medical information of a vehicle occupant of a motor vehicle, and/or of a medical device of the vehicle occupant, may be used more in-depth and individually.

According to the invention, this object is achieved by a method, by a system, and by a motor vehicle having the features according to the respective independent claims.

### BRIEF DESCRIPTION OF FIGURES

FIG. 1 illustrates a diagram of a motor vehicle and an external system for gathering medical information, according to an embodiment.

### DETAILED DESCRIPTION

In a method according to the invention, medical information of a vehicle occupant of a motor vehicle and/or of a medical device of the vehicle occupant is provided. In the method, a signal comprising the medical information is transmitted wirelessly to equipment arranged in the motor vehicle. A significant idea of the invention is that the medical information is transmitted wirelessly from the equipment, especially by means of a communication unit of the equipment, to an apparatus external to the motor vehicle.

Via the method according to the invention, it is possible to use the medical information of the vehicle occupant of the motor vehicle and/or of the medical device of the vehicle occupant comprehensively and in depth. The medical information may thus be analyzed precisely and deeply in the apparatus external to the motor vehicle. Depending on a finding of the analysis or evaluation, for example, an individual and technically founded consequence may then be introduced. The safety of the motor vehicle may thereby be increased.

The medical information is especially a health status of the vehicle occupant and/or an operating status of the medical device. The medical device is in particularly permanently connected with the vehicle occupant. For example, the medical device may thus be designed as an insulin pump and/or a blood sugar measurement device and/or a hearing aid and/or a cardiac pacemaker and/or a prosthesis and/or a dialysis device. The medical device is preferably active equipment having sensors and parameters for fine tuning. The medical device is especially not arranged at the motor vehicle, and thus especially is not a sensor of the motor vehicle. The medical device is thus only located in an interior space of the motor vehicle in the event that the vehicle occupant is located in the interior space of the motor vehicle. It is especially provided that the medical information about the health status of the vehicle occupant and/or the operating status of the medical device is transmitted to the apparatus external to the motor vehicle, thus for example a database which is located outside of the motor vehicle. Following this, the transmitted medical information may be evaluated automatically and/or by a human operator, and measures may be introduced as a consequence. For example, the measures may be a transmission of parameters to the equipment and furthermore to the medical device in order to adapt the operating status of the medical device to the health status of the vehicle occupant. Furthermore, the medical information may be provided by the apparatus external to the motor vehicle so that a rescue service which is called to the motor vehicle and/or the vehicle occupant may already access the medical information during the drive to the motor vehicle, and may accordingly make early preparations. In an emergency, valuable time may hereby be saved. For example, it may also be that a physician, on the basis of the medical information, remotely grants instructions to a medically untrained person present at the location of the motor vehicle. The vehicle occupant may thus likewise be provided with prompt support.

Furthermore, it is advantageous that the health status may especially be established without an explicit action of the vehicle occupant. That means that the vehicle occupant preferably does not notice the transmission of the medical information at all. The vehicle occupant may thus in particular focus on driving the motor vehicle. However, due to the apparatus external to the motor vehicle, the vehicle occupant is at all times connected with a suitable location in order to evaluate and/or to monitor the medical information and/or to transmit it to the suitable location, and possibly to react thereto.

The wireless transmission of the signal with the medical information preferably takes place in the electromagnetic range, thus via radio, and/or in the ultrasound range, thus acoustically, for example with ultrasonic transmitters and receivers. Additionally or alternatively, it may be provided that the medical information is exchanged via speech signals, thus a speech output and a speech detection. For example, the medical device may thus be designed to output the health status and/or the operating status by means of speech or words. The speech may then in turn be detected by the device, for example, and be transmitted to the apparatus external to the motor vehicle. The wireless transmission of the medical information from the equipment to the apparatus external to the motor vehicle may take place using a mobile communications standard, for example. However, a communication device of the motor vehicle that is already present in the motor vehicle, separate from the equipment, may be used for the wireless transmission of the medical information to the apparatus external to the motor vehicle, for example.

It is especially provided that the medical device is connected permanently with the body of the vehicle occupant and/or is designed as medical device implanted into the body of the vehicle occupant. This means that the medical device is not arranged at the motor vehicle, and thus is not designed as a sensor of the motor vehicle. If the vehicle occupant leaves the interior space of the motor vehicle, this especially leads to the situation that the medical device is likewise no longer present in the interior space. For example, the medical device may be designed as an implanted defibrillator and/or cardiac pacemaker and/or insulin pump and/or blood sugar measurement device and/or hearing aid and/or prosthesis and/or dialysis device. The medical device especially has an interface which is compatible with an interface of the equipment in order to transmit the signal with the medical information wirelessly. It is advantageous that the medical device permanently connected with the body of the occupant may provide more precise and continuative medical information about the health status of the vehicle occupant than is enabled with, for example, sensors of the motor vehicle.

Furthermore, it is provided in particular that the apparatus external to the motor vehicle is provided as a database, especially of a medical care institution and/or of a manufacturer of the medical device. The medical care institution may be formed as a hospital and/or a physician practice, for example. Furthermore, it may be provided that the database is provided by a manufacturer of the motor vehicle. For example, the database may be mapped with a relational database model or a non-relational database model. It may also be preferable that the database is transmitted to additional databases in order to be able to provide the medical information simultaneously to multiple sites. For example, the database may also be present in a rescue vehicle. It may also be provided that the medical information in the database is supplemented with historical medical information of the vehicle occupant. The historical medical information may, for example, have been recorded at an earlier point in time than the current medical information transmitted to the apparatus external to the motor vehicle. Due to the historical medical information, the database is more significant, and measures to assist the vehicle occupant may be introduced more effectively.

It is preferably provided that the medical information is evaluated by means of the apparatus external to the motor vehicle, and a finding of the evaluation of the apparatus external to the motor vehicle is transmitted to the equipment, wherein at least one parameter of the medical device is adapted depending on the transmitted finding via transmission of an adaptation signal from the equipment to the medical device. The evaluation of the medical information may take place automatically on the basis of predetermined procedures, for example. Additionally or alternatively, however, the evaluation may also be performed by a person, for example a physician. Depending on the evaluation, a finding may be determined and transferred or transmitted to the equipment. Depending on the transmitted finding, the equipment may now send the adaptation signal to the medical device in order to adapt at least one parameter of the medical device. For example, a setting of the insulin pump may thus be changed so that more or less insulin is dispensed to the vehicle occupant. It is thus advantageous that the parameter of the medical device may be expertly adapted individually and currently on the basis of the medical information present in the apparatus external to the motor vehicle. “Currently” thus means that the parameters may be adapted live or in real time.

Furthermore, it is preferably provided that the medical information is evaluated by means of the apparatus external to the motor vehicle, and a first communication to the vehicle occupant and/or an additional vehicle occupant of the motor vehicle is output in the motor vehicle depending on a finding of the evaluation that is transmitted to the equipment from the apparatus external to the motor vehicle, and in particular a reaction of the vehicle occupant and/or of the additional vehicle occupant to the first communication is determined by the equipment. The first communication to the vehicle occupant and/or the additional vehicle occupant may thus take place on the basis of the transmitted finding of the evaluation. What finding the evaluation has yielded, and for example what consequences and/or action instructions follow from this or what action steps may be introduced, may be indicated to the vehicle occupant, for example in the form of a visual notification. Additionally or alternatively, the first communication may also be output to the additional vehicle occupant who is not permanently connected to the medical device, in order to obtain from the additional vehicle occupant assistance or support for the vehicle occupant having the medical device. A concrete action instruction may thus also be output to the additional vehicle occupant. Via the determination of the reaction, it may be established whether the second communication has been correctly received or understood by the vehicle occupant and/or the additional vehicle occupant. For example, a test question or a test reaction may be queried and determined via the reaction. The test reaction enables the testing of a physical and/or mental status of the vehicle occupant.

It is preferably provided that the first communication is output visually and/or haptically and/or acoustically in the motor vehicle. Visually, the first communication may be output on a screen of the motor vehicle, for example. Haptically, the first communication may be output via a vibration of the steering wheel of the motor vehicle, for example. Acoustically, the first communication is output via a loudspeaker system of the motor vehicle, for example. For example, the first communication may be output acoustically as a warning tone or as a speech output, for example with concrete current action instructions. It is advantageous that the vehicle occupant and/or the additional vehicle occupant may be instructed in various ways, and thus clearly, via the first communication of the transmitted finding.

In a further embodiment, it is preferably provided that the medical information is evaluated by means of the apparatus external to the motor vehicle, and a second communication to a person external to the motor vehicle, especially to a medically trained person, is output by the apparatus external to the motor vehicle depending on the finding of the evaluation. The person external to the motor vehicle may be a physician and/or an emergency medical technician in a rescue vehicle, for example. Additionally or alternatively, for example, the physician may also wait in a hospital for a delivery of the vehicle occupant and, in preparation, may be prepared for the vehicle occupant beforehand with the second communication, for example may be prepared for a pending operation. A well prepared treatment of the vehicle occupant may thereby take place.

In a further embodiment, it is preferably provided that the medical information is evaluated by means of the apparatus external to the motor vehicle, and an intervention in the control of the motor vehicle is executed depending on a finding of the evaluation. The finding of the evaluation may thus be transmitted back to the equipment, for example from the apparatus external to the motor vehicle, and the control of the motor vehicle may thereby be influenced. For example, the intervention in the control of the motor vehicle encompasses a braking of the motor vehicle and/or an acceleration of the motor vehicle and/or a steering of the motor vehicle and/or a travel route proposed by the navigation system of the motor vehicle. All vehicle parameters of the motor vehicle which indirectly or directly relate to a movement of the motor vehicle are thus especially encompassed by the control of the motor vehicle. For example, the motor vehicle may thus be stopped by an emergency braking due to the finding. Additionally or alternatively, a current travel route may also be output to the driver of the motor vehicle, especially the vehicle occupant, via the navigation system in order to navigate the vehicle occupant as quickly as possible to a location at which said occupant may be treated as best possible with regard to their health status. Via the evaluation of the medical information by means of the apparatus external to the motor vehicle, a more precise intervention in the control of the vehicle may be taken than if an evaluation of the medical information were to take place exclusively within the motor vehicle.

Furthermore, it is preferably provided that the vehicle occupant may substitute a necessary transmission of the medical information (for example at home) with the transmission of the medical information to the equipment. For example, it may be provided that the vehicle occupant should retrieve the medical information from the medical device at predetermined points in time, for example once daily. The retrieval of the medical information at the predetermined point in time may thus take place during use of the motor vehicle, and at no additional time during the day must effort be made in order to retrieve or query the medical information. The everyday life of the vehicle occupant may thereby be more efficiently arranged.

Furthermore, the invention relates to a system for providing medical information of a vehicle occupant of a motor vehicle and/or of a medical device of the vehicle occupant, which system has a motor vehicle having an internal equipment or equipment, and with an apparatus external to the motor vehicle, wherein the system is designed such that a signal comprising the medical information is wirelessly transferred to the equipment arranged in the motor vehicle, and the medical information is transmitted wirelessly from the equipment to the apparatus external to the motor vehicle. For this, the system is designed to execute a method according to the invention or an advantageous embodiment thereof.

A motor vehicle according to the invention, having an internal equipment which is designed for wireless communication with a medical device of a vehicle occupant and for wireless communication with an apparatus external to the motor vehicle for wireless transmission of a signal comprising a medical information [sic].

The preferred embodiments proposed with reference to the method according to the invention, and their advantages, accordingly apply to the system according to the invention and to the motor vehicle according to the invention.

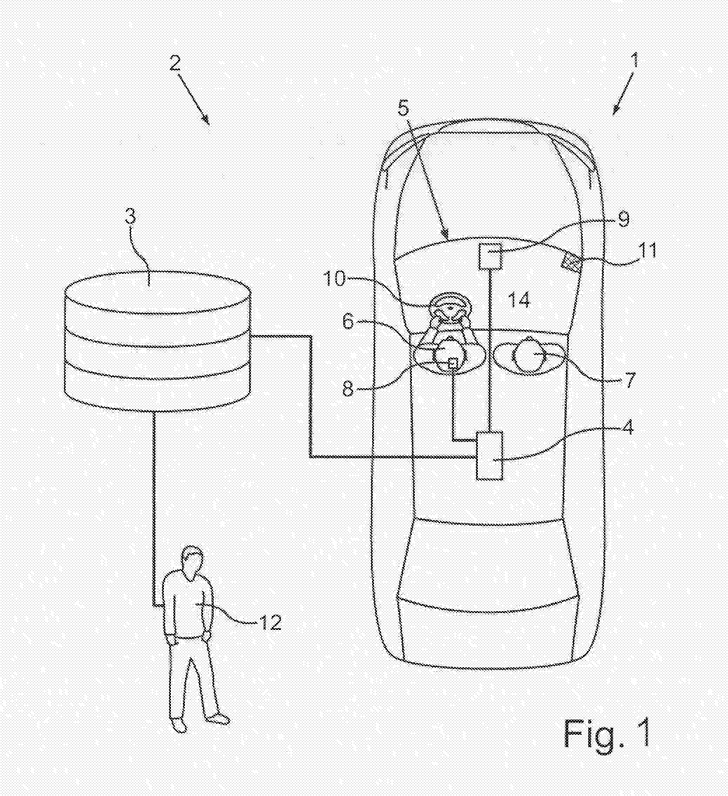

Additional features of the invention result from the claims, FIG. 1 and the FIG. 1 description. All features and feature combinations cited in the specification, as well as the features and feature combinations subsequently cited in the FIG. 1 description and/or shown alone in FIG. 1, are usable not only in the respective indicated combination but also in other combinations or alone, without leaving the scope of the invention. Thus, embodiments of the invention are also to be considered as being comprised and disclosed, which are not explicitly shown and explained in FIG. 1, but which can result and can be generated from the described embodiments by separate feature combinations. Embodiments and feature combinations are also to be considered as being disclosed, which thus do not have all the features of an originally formulated independent claim.

The invention is now explained in detail using a preferred exemplary embodiment, as well as with reference to the attached drawing.

FIG. 1 thereby shows a schematic depiction of an exemplary embodiment of a motor vehicle according to the invention, having an internal equipment, and an apparatus external to the motor vehicle.

FIG. 1 shows a schematic depiction of a motor vehicle 1 and of a system 2. The system 2 comprises the motor vehicle 1 and an apparatus 3 external to the motor vehicle. The apparatus 3 external to the motor vehicle is preferably designed as a database and, for example, is stored at a hospital and/or at a physician practice and/or at a manufacturer of the motor vehicle 1.

The motor vehicle 1 comprises equipment 4. Located in an interior space 5 of the motor vehicle 1 are a vehicle occupant 6 and an additional vehicle occupant 7. According to the exemplary embodiment, the vehicle occupant 6 is provided as the driver of the motor vehicle 1, and the additional vehicle occupant 7 is provided as the passenger of the motor vehicle 1. However, it may also be that the additional vehicle occupant 7 is the driver of the motor vehicle 1, or that neither the vehicle occupant 6 nor the additional vehicle occupant 7 are drivers of the motor vehicle 1.

A medical device 8 is arranged on or in the vehicle occupant 6. For example, the medical device 8 may be designed as an insulin pump and/or a blood sugar measurement equipment and/or a hearing aid and/or a cardiac pacemaker and/or a prosthesis and/or a dialysis device and/or an implanted defibrillator. The medical device 8 and the equipment 4 respectively possess interfaces and are designed to exchange medical information with one another. Medical information may thus be transmitted wirelessly from the medical device 8 to the equipment 4. For example, the wireless transfer takes place by means of radio technology and/or by means of acoustic transmission, for example by means of ultrasound. The medical information is especially characterized by a health status of the vehicle occupant 6 and/or an operating status of the medical device 8.

According to the exemplary embodiment, the equipment 4 is arranged centrally at the motor vehicle 1; however, the arrangement of the equipment 4 in various places at the motor vehicle 1 is possible, but preferably so that a communication or a transfer of the medical information from the medical device 8 to the equipment 4 is possible wirelessly or without cabling.

It is now provided that the medical information is transmitted wirelessly from the equipment 4 to the apparatus 3 external to the motor vehicle. For example, this may be triggered manually by the vehicle occupant 6 and/or be implemented automatically by the equipment 4, for example at regular intervals. The medical information may now be evaluated by means of the apparatus 3 external to the motor vehicle. A finding is provided as a result of the evaluation. For example, the evaluation may take place automatically via predetermined workflow schemes and/or may be performed by a person. For example, the person may be a physician and/or a person who is otherwise medically trained for the specific need of the vehicle occupant 6.

The finding may now be transferred to the equipment 4 in order to, for example, adapt a parameter of the medical device 8 depending on the transmitted finding. For this, in particular an adaptation signal is sent from the equipment 4 to the medical device 8. In particular, first the finding is transferred from the apparatus 3 external to the motor vehicle to the equipment 4, and the adaptation signal having at least one parameter of the medical device 8 is subsequently sent from the equipment 4 to the medical device. The parameter may be encompassed by the finding or, however, may already be stored in the equipment 4.

However, the finding of the evaluation may also be transmitted to the equipment 4 from the apparatus 3 external to the motor vehicle in order to output a first communication via the equipment 4, for example at a display unit 9 of the motor vehicle 1. For example, the first communication is a warning notice and/or a treatment instruction. The first communication may thus be output visually at the display unit 9, for example. Additionally or alternatively, the first notification may be a haptic output at a steering wheel 10 of the motor vehicle 1, for example. The haptic output may, for example, take place via a vibration or a shaking of the steering wheel 10. Furthermore, the first notification may additionally or instead be output acoustically via a loudspeaker 11 of the motor vehicle 1. The first communication may thus be indicated to the vehicle occupant 6 and/or the additional vehicle occupant 7 via the display unit 9 and/or the steering wheel 10 and/or the loudspeaker 11. Furthermore, for example, a reaction of the vehicle occupant 6 and/or of the additional vehicle occupant 7 to the first communication may be determined. For example, it may thus be checked whether there has been a reaction to the first communication. In the event that no reaction takes place, or a different reaction than expected is made by the vehicle occupant 6, this may be transmitted from the equipment 4 to the apparatus 3 external to the motor vehicle and enter into a subsequent evaluation of the medical data. A check may thus be made as to whether the first communication from the vehicle occupant 6 and/or from the additional vehicle occupant 7 has been registered or correctly registered. Furthermore, it may thereby be determined whether the vehicle occupant 6 is conscious, for example.

The finding of the evaluation may additionally or alternatively be transmitted as a second communication to a person 12 external to the motor vehicle. The second communication includes, for example, detailed information about the current health status of the vehicle occupant 6. For example, the person 12 external to the motor vehicle may be a medically trained person such as a physician or an emergency medical technician. In an embodiment, an instruction for treatment of the vehicle occupant 6 may be provided by the person 12 external to the motor vehicle to the additional vehicle occupant 7. However, based on the medical information the person 12 external to the motor vehicle may also prepare for a possibly pending rescue operation in order to implement the treatment of the vehicle occupant 6 as promptly as possible and effectively after arriving on site.

Additionally or alternatively, the finding which is transferred from the apparatus external to the motor vehicle to the equipment 4 may also be used in order to execute an intervention in the control of the motor vehicle 1. The intervention in the control of the motor vehicle 1 may, for example, be a braking, especially an emergency braking, or an acceleration and/or a steering of the motor vehicle 1. However, via the control, a control command may also be granted to a control unit of the motor vehicle 1 in numerous ways. For example, a distance-regulating cruise control (ACC—Adaptive Cruise Control) of the motor vehicle 1 may thus also be adapted depending on the finding. Based on the finding, it is thus known that the vehicle occupant 6, especially the driver of the motor vehicle 1, is limited in his perception of the current traffic situation; a minimum distance of the motor vehicle 1 from the preceding motor vehicle may thus be increased. The medical information, in the form of the health status of the vehicle occupant 6, may thus be characterized by a low blood sugar level, for example. The evaluation of this medical information may then lead to the situation that symptoms must be reckoned with, such as a blurring of the consciousness of the vehicle occupant 6. Finally, the finding which is transferred to the equipment 4 may lead to the situation that the first communication and/or the second communication is output, and/or the intervention in the control of the motor vehicle is executed.

Furthermore, an adaptation of the output (from a navigation system of the motor vehicle 1, for example) navigation path or navigation suggestion of the motor vehicle 1 may be executed via the intervention in the control of the motor vehicle 1. The motor vehicle 1 may thus, for example, be guided in the fastest way to a hospital or a medical care facility.

Furthermore, an emergency call based on the finding of the evaluation of the medical information may be made, for example to an emergency call center, via the intervention in the control of the motor vehicle 1 and/or by the equipment 4 itself. For this purpose, the current location of the motor vehicle 1, which has been determined, for example, by means of a GNSS receiver (GNSS—Global Navigation Satellite System) of the motor vehicle 1, can also be considered.

It may additionally be provided that the apparatus 3 external to the motor vehicle also comprises historical medical information of the vehicle occupant 6. For example, the historical medical information may provide a complete etiopathology of the vehicle occupant 6, for example of the preceding days or of the preceding months. In the apparatus 3 external to the motor vehicle, for example, the historical medical information may then be merged with the medical information or the current medical information which is transmitted by the equipment 4 to the apparatus 3 external to the motor vehicle.

1-11. (canceled)

12. A method for providing medical information of a vehicle occupant of a motor vehicle and of a medical device of the vehicle occupant, comprising:
receiving wirelessly, by equipment arranged in the motor vehicle, a signal comprising the medical information; and
transmitting wirelessly, from the equipment arranged in the motor vehicle, the medical information to an apparatus external to the motor vehicle,
wherein the signal comprising the medical information is acoustically transmitted wirelessly in the range of soundwaves.

13. The method of claim 12, wherein the medical device is permanently connected with the body of the vehicle occupant and/or is designed as a medical device implanted in the body of the vehicle occupant.

14. The method of claim 12, wherein the apparatus external to the motor vehicle comprises a database of a medical care facility or of a manufacturer of the medical device.

15. The method of claim 12, comprising:
outputting, by the equipment, a finding to the vehicle occupant or to an additional vehicle occupant of the motor vehicle based on the finding of an evaluation, and
determining, by the equipment, a reaction to the communication by the vehicle occupant or the additional vehicle occupant,
wherein the medical information is evaluated by the apparatus external to the motor vehicle and a communication indicating a finding based on the evaluated medical information is transmitted from the apparatus external to the motor vehicle to the equipment arranged in the motor vehicle.

16. The method of claim 15, wherein the finding is output visually and/or haptically and/or acoustically.

17. The method of claim 12, wherein the medical information is evaluated by the apparatus external to the motor vehicle to obtain a finding, and a communication indicating a preparation for the vehicle occupant is output by the apparatus external to the motor vehicle to a physician at a hospital based on the finding.

18. The method of claim 12, wherein the medical information is evaluated by the apparatus external to the motor vehicle to obtain a finding, and an intervention in a control of the motor vehicle is executed based on a finding of the evaluation of the medical information.

19. The method of claim 12, wherein the signal comprising the medical information is acoustically transferred wirelessly in the range of ultrasound.

20. A system for providing a medical information of a vehicle occupant of a motor vehicle, and/or of a medical device of the vehicle occupant, comprising:
internal equipment arranged in the motor vehicle, configured to:
receive wirelessly, from the medical device, a signal comprising the medical information arranged in the motor vehicle, wherein the signal comprising the medical information is acoustically transmitted wirelessly in a range of soundwaves; and
transmit wirelessly the medical information to an apparatus external to the motor vehicle.

21. The system of claim 20, further comprising the apparatus external to the motor vehicle, configured to:
evaluate the medical information; and
transmit a finding of the evaluation to the internal equipment of the motor vehicle,
and the medical device is further configured to adapt at least one parameter of the medical device by receiving an adaptation signal from the internal equipment.

22. The system of claim 20, wherein the medical device is connected with the body of the vehicle occupant or is designed as a medical device implanted in the body of the vehicle occupant.

23. The system of claim 20, wherein the apparatus external to the motor vehicle is a database of a medical care facility or of a manufacturer of the medical device.

24. The system of claim 20, further comprising the apparatus external to the motor vehicle, configured to:
evaluate the medical information; and
output, to a hospital facility, a communication indicating a preparation for the vehicle occupant based on a finding of the evaluation.

25. The system of claim 20, further comprising the apparatus external to the motor vehicle, configured to:
evaluate the medical information to obtain a finding; and
execute an intervention in a control of the motor vehicle based on the finding.

26. A motor vehicle, comprising:
internal equipment, configured to:
wirelessly receive a signal comprising medical information from medical equipment of a vehicle occupant; and
wirelessly transmit another signal comprising the medical information to an apparatus external to the motor vehicle;
wherein the signal comprising the medical information is acoustically transferred wirelessly in the range of soundwaves.
